(19) United States
(12) Patent Application Publication
(10) Pub. No.: US 2019/0117559 A1
(43) Pub. Date: Apr. 25, 2019

(54) METHOD OF TREATING INFLAMMATION AND PROMOTING WOUND HEALING

(71) Applicant: Sang Pacific Enterprises Pty Ltd.,, SPRINGFIELD , Queensland (AU)

(72) Inventor: Sang Ha, SPRINGFIELD QLD (AU)

(73) Assignee: Sang Pacific Enterprises Pty Ltd.,, SPRINGFIELD , Queensland (AU)

(21) Appl. No.: 16/095,106

(22) PCT Filed: Apr. 21, 2017
(86) PCT No.: PCT/AU2017/050367

§ 371 Date: Oct. 19, 2018

(30) Foreign Application Priority Data
Apr. 21, 2016 (AU) 2016901486

(51) Int. Cl. A61K 9/00; A61K 36/00; A61K 47/44; A61K 36/28; A61K 31/355; A61K 31/16; A61K 31/12; A61K 36/23; A61K 36/53; A61K 36/738; A61K 36/328; A61K 36/752; A61P 29/00

(52) U.S. Cl. CPC A61K 9/0014; A61K 36/00; A61K 47/44; A61K 36/28; A61K 31/355; A61K 31/16; A61P 29/00; A61K 36/23; A61K 36/53; A61K 36/738; A61K 36/328; A61K 36/752; A61K 31/12

(57) ABSTRACT

The present invention relates to a composition for treating inflammation and/or promoting wound healing comprising macadamia oil, beeswax and shea butter as the major active components. The composition has efficacy in the treatment of a range of skin conditions.

### FIELD OF THE INVENTION

The invention relates to the field of medical treatment. More particularly, this invention relates to the treatment of inflammation and the promotion of wound healing.

### BACKGROUND TO THE INVENTION

Any reference to background art herein is not to be construed as an admission that such art constitutes common general knowledge in Australia or elsewhere.

Inflammation is a non-specific reaction mounted by the immune system in response to a perceived injury or threat. While associated with infection, inflammation occurs in response to many types of injury, including physical trauma, burns (e.g., from radiation, heat or corrosive materials), chemical or particulate irritants, bacterial or viral pathogens, and localized oxygen deprivation (ischemia). Inflammation is also associated with autoimmune diseases and allergic reactions. Inflammation includes the classic symptoms of redness, heat, swelling, and pain, and may be accompanied by decreased function of the inflamed organ or tissue.

While a number of methods for treating inflammation are known, all of them have limitations, particularly with regard to general efficacy and safety profile of the therapy for application in the long term.

Inflammation is the body's natural defence to infection when trauma is received. It rapidly provides a barrier to entry against infectious agents and promotes a return to the tissue's natural condition. Inflammation is, of course, a key phase in the wound healing process. Many existing treatments simply provide a de facto barrier to infectious agents but do not of themselves actively promote wound healing and, indeed, in often cases will delay the process due to the lack of air exposure or to the wound being further damaged on removal of the barrier treatment. Thus, there is a need for new methods for reducing, alleviating and/or preventing inflammation and of promoting wound healing, associated with a variety of causes.

### SUMMARY OF INVENTION

The present invention is directed to methods and compositions for treating and/or preventing inflammation and/or diseases or conditions associated with inflammation and/or for promoting wound healing.

The present invention is predicated, at least in part, on the finding that a composition comprising a number of natural components, i.e. those found occurring in nature, can provide a surprisingly effective reduction of inflammation and promotion of the wound healing process while maintaining an advantageous safety profile.

In a first aspect, the invention resides in a composition for treating inflammation and/or promoting wound healing comprising macadamia oil, beeswax and shea butter as the major active components.

In a second aspect, the invention resides in a method of treating inflammation and/or promoting wound healing in a subject, the method including the step of administering to the subject a therapeutically effective amount of a composition comprising macadamia oil, beeswax and shea butter as the major active components.

In a third aspect, the invention resides in a method of formulating the composition of the first aspect including the steps of mixing macadamia oil, beeswax and shea butter to form the composition wherein the macadamia oil, beeswax and shea butter form the major active components.

In a fourth aspect, the invention resides in the use of a composition comprising macadamia oil, beeswax and shea butter for the preparation of a medicament for use in treating inflammation and/or promoting wound-healing.

The various features and embodiments of the present invention, referred to in individual sections above apply, as appropriate, to other sections, mutatis mutandis. Consequently features specified in one section may be combined with features specified in other sections as appropriate.

Further features and advantages of the present invention will become apparent from the following detailed description.

### BRIEF DESCRIPTION OF THE DRAWINGS

To assist in understanding the invention, the invention will be described by way of example only with reference to the accompanying drawings, in which:

FIG. 1 shows the hand of a patient shortly after a burn; and

FIG. 2 shows the same hand shown in FIG. 1 after the application of the present invention

Further features and advantages of the present invention will become apparent from the following detailed description.

### DETAILED DESCRIPTION

### Definitions

In this patent specification, the terms ‘comprises’, ‘comprising’, ‘includes’, ‘including’, or similar terms are intended to mean a non-exclusive inclusion, such that a method or composition that comprises a list of elements does not include those elements solely, but may well include other elements not listed.

As used in this specification the indefinite articles “a” and “an” may refer to one entity or a plurality of entities (e.g. components) and are not to be read or understood as being limited to a single entity.

As generally used herein, the terms “administering”, “administration” or “administered” describe the introduction of the recited components or composition to a mammal by a particular route or vehicle. Routes of administration may include, for example, topical, although without limitation thereto.

The terms “treat”, “treatment” or treating”, as used herein, refers to a therapeutic intervention that ameliorates a sign or symptom of inflammation after it has begun to develop or which assists in efficient wound healing. The term “ameliorating,” with reference to inflammation, refers to any observable beneficial effect of the treatment. The beneficial effect can be determined using any methods or standards known to the ordinarily skilled artisan.

By “prevent”, “preventing” or “preventative” is meant prophylactically administering the compound or composition to a mammal who does not exhibit signs or symptoms of inflammation, but who is expected or anticipated to likely exhibit such signs or symptoms in the absence of prevention. Preventative treatment may at least lessen or partly ameliorate expected symptoms or signs.

By “reducing”, as in reducing inflammation in a subject, is meant a lessening or shortening of a symptom, aspect, or characteristic associated with inflammation (e.g., redness, heat, swelling, and/or pain), or of the length of time a subject experiences a symptom, aspect, or characteristic associated with inflammation. Such reducing need not be absolute to be beneficial to the subject.

By “alleviating”, as in alleviating inflammation in a subject, is meant a reduction in the severity or seriousness of a symptom, aspect, or characteristic associated with inflammation (e.g., redness, heat, swelling, and/or pain). Such alleviating need not be absolute to be beneficial to the subject. Reduction and/or alleviation of inflammation in a subject can be determined using any methods or standards known to the ordinarily skilled artisan, including both qualitative and quantitative methods and standards.

The term “subject” includes both human and veterinary subjects. For example, administration of the composition to a subject can include administration to a human subject or a veterinary subject. Preferably, the subject is a human. However, therapeutic uses according to the invention may also be applicable to mammals such as domestic and companion animals, performance animals such as horses, livestock, and laboratory animals.

The term “therapeutically effective amount” describes a quantity of the composition of the first aspect sufficient to achieve a desired effect in a subject being treated. For example, this can be the amount of a composition comprising macadamia oil, beeswax and shea butter as the major active components necessary to reduce, alleviate and/or prevent inflammation and/or to promote wound healing. In some embodiments, a “therapeutically effective amount” is sufficient to reduce or eliminate a symptom of inflammation and/or to promote wound healing. In other embodiments, a “therapeutically effective amount” is an amount sufficient to achieve a desired biological effect, for example an amount that is effective to decrease redness, heat, swelling, and/or pain associated with inflammation and/or wound healing. The “therapeutically effective amount” will vary, in a manner which would be understood by a person of skill in the art, with patient age, sex, weight etc.

As used herein, “inflammation” refers to the well-known localised response to various types of injury or infection, which is characterised by redness, heat, swelling, and pain, and often also including dysfunction or reduced mobility. Inflammation represents an early defence mechanism to contain an infection and prevent its spread from the initial focus. Major events in inflammation include dilation of capillaries to increase blood flow, changes in the microvasculature structure, leading to escape of plasma and proteins and leukocytes from the circulation, and leukocyte emigration from the capillaries and accumulation at the site of injury or infection.

The term “about” as used herein in relation to the amount of certain components, means that the amount is nominally the number following the term “about” but the actual amount may vary from this precise amount to an unimportant degree.

Unless defined otherwise, all technical and scientific terms used herein have the same meaning as would be commonly understood by those of ordinary skill in the art to which this invention belongs.

In a first aspect, the invention resides in a composition for treating inflammation and/or promoting wound healing comprising macadamia oil, beeswax and shea butter as the major active components.

The term “major active components” as used herein, in relation to the relative amount of macadamia oil, beeswax and shea butter, in the composition refers to the major active components making up greater than about 65%, suitably greater than about 80% and preferably greater than about 90% by weight amount of the composition.

The % by weight amount of the macadamia oil in the composition is suitably from about 65% to about 95%, more suitably about 70% to about 90%, and preferably about 75% to about 85%, including values of about 75%, 76%, 77%, 78%, 79%, 80%, 81%, 82%, 83%, 84% and 85%. Most preferably, the % by weight amount of the macadamia oil in the composition is about 80%.

The term “macadamia oil” as used herein refers to any oil that is derived from the macadamia nut including purified and/or refined derivatives of macadamia nuts. Macadamia oil may be derived from Macadamia intergrifolia. Without being bound to any particular constituent, macadamia oil may include one or more chemical compounds such as, oleic acid, palmitoleic acid, linoleic acid, and/or α-linolenic acid. These chemical compounds can be produced synthetically or derived from another source including essential oils.

The % by weight amount of the beeswax in the composition is suitably from about 5% to about 15%, preferably from about 6% to about 14%, and more preferably about 7% to about 13%, including values of about 7%, 8%, 9%, 10%, 11%, 12% and 13%. Most preferably, the % by weight amount of the beeswax in the composition is about 10%.

The term “beeswax” as used herein refers to the wax produced by honey bees including purified and/or refined derivatives of the wax produced by honey bees. Beeswax may be derived from the wax produced by any of the genus Apis. Without being bound to any particular constituent, beeswax may include one or more chemical compounds such as, palmitate, palmitoleate, and/or oleate esters of long chain aliphatic alcohols. These chemical compounds can be produced synthetically or derived from another source.

The % by weight amount of the shea butter in the composition is suitably from about 1% to about 8%, preferably from about 1.5% to about 7%, and more preferably about 2% to about 5%, including values of about 2%, 3%, 4% and 5%. Most preferably, the % by weight amount of the shea butter in the composition is about 4%.

The term “shea butter” as used herein refers to the oil that is derived from the nut of the African shea tree including purified and/or refined derivatives of the nut of the African shea tree. Without being bound to any particular constituent, shea butter may include one or more chemical compounds such as, oleic acid, stearic acid, linoleic acid, and/or palmitic acid. These chemical compounds can be produced synthetically or derived from another source including essential oils.

The composition of the first aspect of the invention may further comprise at least one additional component selected from the group consisting of vitamin E, calendula oil, vitamin B5, benzoin oil, carrot seed oil, patchouli oil, geranium oil, lavender oil, sandalwood oil, rose oil, myrrh oil and citrus oil. It will be appreciated that these components can include one or more known chemical compounds and that these chemical compounds can be produced synthetically or derived from a natural source.

In one embodiment, the composition of the first aspect comprises at least 2, or at least 3, or at least 4, or at least 5, or at least 6, or at least 7, or at least 8, or at least 9 of the additional components.

In one embodiment all of the additional components are present.

The % by weight amount of the one or more of the “additional components” in the composition is suitably less than about 15%, more suitably less than about 10%, preferably less than about 8%, more preferably less than about 6%, even more preferably less than about 5% and most preferably less than about 2% by weight of the composition. In one embodiment, the % by weight amount of the one or more of the “additional components” in the composition is less than about 1%.

In one embodiment, the composition of the first aspect consists of macadamia oil, beeswax and shea butter as the major active components.

In another embodiment, the composition of the first aspect consists essentially of macadamia oil, beeswax and shea butter as the major active components. As used herein, the term ‘consists essentially of’ means that the composition can include additional elements, but only if those additional elements do not materially affect the workings of the composition.

In one embodiment, the composition of the first aspect comprises macadamia oil, beeswax, shea butter, vitamin E, calendula oil, vitamin B5, benzoin oil, carrot seed oil, patchouli oil, geranium oil, lavender oil, sandalwood oil, rose oil, myrrh oil and citrus oil.

In one embodiment, the composition of the first aspect consists of, or alternatively consists essentially of, macadamia oil, beeswax, shea butter, vitamin E, calendula oil, vitamin B5, benzoin oil, carrot seed oil, patchouli oil, geranium oil, lavender oil, sandalwood oil, rose oil, myrrh oil and citrus oil.

In one embodiment, the composition of the first aspect comprises macadamia oil in the range of about 70% to about 90% by weight amount in the composition, beeswax in the range of about 7% to about 13% by weight amount in the composition, shea butter in the range of about 2% to about 5% by weight amount in the composition, and two or more of the additional components in less than or about 2% by weight amount in the composition.

In another embodiment, the composition of the first aspect comprises macadamia oil in about 80% by weight amount of the composition, beeswax in about 10% by weight amount of the composition, shea butter in about 4% by weight amount of the composition, and three or more additional components in less than about 2% by weight amount of the composition.

In a preferred embodiment, the composition of the first aspect comprises macadamia oil in about 80% by weight amount of the composition, beeswax in about 10% by weight amount of the composition, shea butter in about 4% by weight amount of the composition, vitamin E in about 1.8% by weight amount of the composition, calendula oil in about 0.9% by weight amount of the composition, vitamin B5 in about 0.9% by weight amount of the composition, benzoil oil in about 0.7% by weight amount of the composition, carrot seed oil in about 0.4% by weight amount of the composition, patchouli in about 0.4% by weight amount of the composition, geraniuim oil in about 0.4% by weight amount of the composition, lavender oil in about 0.4% by weight amount of the composition, sandalwood oil in about 0.4% by weight amount of the composition, rose oil in about 0.4% by weight amount of the composition, myrrh oil in about 0.4% by weight amount of the composition and citrus oil in about 0.3 by weight amount of the composition.

It is advantage of the present invention that no further diluents or excipients are required other than the components mentioned above. However, depending of the mode of administration or formulation and manufacture considerations it may, at times, be useful to include one or more carriers, excipients, diluents and the like as are known in the art.

Any carrier or diluent, or other excipients, will depend on the route of administration, and again the person skilled in the art will readily be able to determine the most suitable formulation for each particular case.

Frequently used carriers or auxiliaries include but are not limited to other oils, sugars, talc, milk protein, gelatin, starch, vitamins, cellulose and its derivatives, animal and vegetable oils, polyethylene glycols and solvents, such as sterile water, alcohols, glycerol and polyhydric alcohols. Preservatives include antimicrobials, anti-oxidants, and chelating agents. Other pharmaceutically acceptable carriers include aqueous solutions, non-toxic excipients, including salts, preservatives, buffers and the like, as described, for instance, in Remington's Pharmaceutical Sciences, 20th ed. Williams & Wilkins (2000) and The British National Formulary 43rd ed. (British Medical Association and Royal Pharmaceutical Society of Great Britain, 2002; http://bnf.rhn.net), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed., 1985).

In one embodiment, the composition further comprises a preservative. The preservative may not be necessary if the composition is to be used shortly after manufacture but is preferred for storage. The preservative is a substance that is added to the composition to prevent microbial growth and undesirable chemical changes. The preservative can be naturally derived, or a synthetically produced.

Diluents may include one or more of microcrystalline cellulose, lactose, mannitol, calcium phosphate, calcium sulfate, kaolin, dry starch, powdered sugar, and the like. Binders may include one or more of povidone, starch, stearic acid, gums, hydroxypropylmethyl cellulose and the like. Solvents may include one or more of ethanol, methanol, isopropanol, chloroform, acetone, methylethyl ketone, methylene chloride, water and the like. Buffers may include phosphate buffers, borate buffers and carbonate buffers, although without limitation thereto. Fillers may include one or more gels inclusive of gelatin, starch and synthetic polymer gels, although without limitation thereto. Coatings may comprise one or more of film formers, solvents, plasticizers and the like. Suitable film formers may be one or more of hydroxypropyl methyl cellulose, methyl hydroxyethyl cellulose, ethyl cellulose, hydroxypropyl cellulose, povidone, sodium carboxymethyl cellulose, polyethylene glycol, acrylates and the like. Suitable solvents may be one or more of water, ethanol, methanol, isopropanol, chloroform, acetone, methylethyl ketone, methylene chloride and the like. Plasticizers may be one or more of propylene glycol, castor oil, glycerin, polyethylene glycol, polysorbates, and the like.

Reference is made to the Handbook of Excipients 6^{th} Edition, Eds. Rowe, Sheskey & Quinn (Pharmaceutical Press), which provides non-limiting examples of excipients which may be useful according to the invention.

It will be appreciated that the choice of pharmaceutically acceptable carriers, diluents and/or excipients will, at least in part, be dependent upon the mode of administration of the formulation. By way of example only, the composition may be in the form of a topical solution, lotion, shake solution, cream, ointment, gel, foam, transdermal patch, powder, solid, sponge, tape, vapour, paste, or balm or any other form that is effective and safe for administration.

The pharmaceutical compositions are preferably prepared and administered in certain forms. Certain preferred forms include gels, balms and oils.

In a second aspect, the invention resides in a method of treating inflammation and/or promoting wound healing in a subject, the method including the step of administering to the subject a therapeutically effective amount of a composition comprising macadamia oil, beeswax and shea butter as the major active components.

The composition of the second aspect is as described herein for any one or more embodiments of the first aspect.

In one embodiment, the composition employed in the second aspect further includes at least one additional component selected from the group consisting of vitamin E, calendula oil, vitamin B5, benzoin oil, carrot seed oil, patchouli oil, geranium oil, lavender oil, sandalwood oil, rose oil, myrrh oil and citrus oil.

In one embodiment, the inflammation and wounds being treated are caused by physical trauma, burns, irritants, bacteria, pathogens, acne, stings, and irritants. It will be understood by a person skilled in that art that the list provided is not an exhaustive list, and the wound and/or inflammation can be caused by a number of other factors.

The composition may be administered by any suitable route, for example topically, and the person skilled in the art will readily be able to determine the most suitable route and effective amount for the condition to be treated. Preferably, the composition is a topical composition. Preferably, the step of administering the composition is a step of topical administration.

The amount of the composition prescribed to a subject may vary depending on manner of administration, nature and severity of the symptoms, age and body weight of the subject. Under certain circumstances, however, higher or lower daily amounts of the composition may be appropriate. The administration of the composition can be carried out both by single administration in the form of an individual application or else several smaller applications and also by multiple applications at specific intervals or may be given in an extended, depot or slow release format.

The composition may additionally be combined with other compositions or compounds to provide an operative combination or co-treatment. It is intended to include any chemically compatible combination of pharmaceutically-active agents, as long as the combination does not negatively impact upon the activity of the metals of this invention.

Preferred embodiments of the composition are in the form of topical solution, lotion, shake solution, cream, ointment, gel, foam, transdermal patch, powder, solid, sponge, tape, vapour, paste, or balm. Preferably, the composition is a substantially homogeneous cream or lotion.

In a third aspect, the invention resides in a method of formulating the composition of the first aspect including the steps of mixing macadamia oil, beeswax and shea butter to form the composition wherein the macadamia oil, beeswax and shea butter form the major active components.

The composition of the third aspect is as described herein for any one or more embodiments of the first aspect.

In one embodiment, the method of formulating the composition of the first aspect further includes adding and mixing of at least one additional component selected from the group consisting of vitamin E, calendula oil, vitamin B5, benzoin oil, carrot seed oil, patchouli oil, geranium oil, lavender oil, sandalwood oil, rose oil, myrrh oil and citrus oil.

In an embodiment, the method of formulating the composition of the first aspect further includes adding and mixing a preservative.

A vessel use for the mixing operation is a chemically resistant vessel which does not react with the composition and has a variable speed stirring. The constituents can be added in any order and stirred for a predetermined amount of time after the addition of each constituent.

Similarly, additional components may be added to give the composition in the form of a topical solution, lotion, shake solution, cream, ointment, gel, foam, transdermal patch, powder, solid, sponge, tape, vapour, paste, or balm. These additional components will be known to a person skilled in the art.

The various features and embodiments of the present invention, referred to in individual sections above apply, as appropriate, to other sections, mutatis mutandis. Consequently features specified in one section may be combined with features specified in other sections as appropriate.

The following examples are provided by way of illustration and are in no way limiting upon the scope of the invention.

### EXPERIMENTAL

### Materials and Methods

### Example 1—Sample Composition

A composition for treating inflammation and/or promoting wound healing comprising macadamia oil, beeswax and shea butter as the major active components was prepared containing the relative amounts of ingredients shown in Table 1 below. The composition was prepared by the process described in Example 2.

TABLE 1
Sample Composition

| Component | Amount (mL) |
|---|---|
| Macadamia Oil | 2250 |
| Beeswax | 260 |
| Shea Butter | 108.53 |
| Vitamin E | 50 |
| Calandula Oil | 25 |
| Vitamin B5 | 25 |
| Benzoin Oil | 18.75 |
| Carrot Seed Oil | 12.5 |
| Patchouli Oil | 12.5 |
| Geranium Oil | 10 |
| Lavender Oil | 10 |
| Sandalwood Oil | 10 |
| Rose Oil | 10 |
| Myrrh Oil | 10 |
| Citrus Oil | 7.5 |
| Total | 2819.78 |

### Example 2—Example Manufacturing Process

The following working steps were taken to produce a composition for treating inflammation and/or promoting wound healing with the relative amounts as set out in table 1:
a. weigh out and add beeswax to a suitably sized vessel and mix;
b. weigh out and add shea butter to the beeswax while mixing;
c. heating the vessel until the beeswax has melted;
d. weigh out and add macadamia oil to the above ingredients while mixing;
e. stirring at a temperature of 40° C. until all of the above ingredients are evenly mixed;
f. weigh out and add the additional components to the above ingredients;
g. stirring until all of the above ingredients are evenly dispersed and a uniform consistency is obtained; and
h. filling suitably sized containers with the composition.

### Patient Data

A number of patients suffering from wounds and/or inflammation, with different symptoms, were treated with the composition of Table 1. Each patient was allowed to apply the composition to the affected areas and the results were monitored.

### Subject A

Subject A
Gender: Female
Age: 51
Skin: Oriental
Symptoms: Cold Sores
Treatment: The composition was applied liberally to the affected area at night.
Results: The cold sores healed completely by morning without scarring.

### Subject A—Trial 2

Subject A
Gender: Female
Age: 51
Skin: Oriental
Symptoms: Blistering of skin, redness of skin and discomfort (adverse reaction to laser therapy treatment)
Treatment: Daily application of the composition was applied liberally to the affected area.
Results: The symptoms were less visible after application of the composition and the symptoms disappeared completely after two weeks of repeated application. Subject A also noted that the discomfort experienced immediately after laser therapy was alleviated within a few hours of applying the composition.

### Subject B

Subject B
Gender: Female
Age: 48
Skin: Caucasian
Symptoms: Blistering of the skin and pimples (adverse reaction to laser therapy treatment)
Treatment: The composition was applied twice a day to the affected area. The composition was applied liberally to the affected areas and thinly to the unaffected areas.
Results: Subject B experienced reduced redness of the skin and scarring after using the composition.

### Subject C

Subject C
Gender: Female
Age: 60
Skin: Caucasian
Symptoms: Blistering of the skin, redness and dryness of the skin (adverse reaction to laser therapy treatment)
Treatment: The composition was applied twice a day two days a week to the affected area. The composition was applied thinly to the affected areas.
Results: Subject C experienced moisturization of the skin and notes that her “skin was moist and well nourished”.

### Subject C—Trial 1

Subject C
Gender: Female
Age: 60
Skin: Caucasian
Symptoms: Chafed lips (“windburn”)
Treatment: The composition was applied to the affected area.
Results: Reduced severity of the symptoms, and Subject C noted that her “lips were softer, hydrated and the composition provided instant relief”.

### Subject D

Subject D
Gender: Male
Age: 52
Skin: Caucasian
Symptoms: Sun spots, redness of skin and sun damage on face and neck.
Treatment: The composition was applied to the affected area. The composition was applied thinly to the affected areas.
Results: The redness of skin and sun damage disappeared after two applications.

### Subject E

Subject E
Gender: Female
Age: 29
Symptoms: Pimples (acne)
Treatment: The composition was applied three times a day to the affected area. The composition was applied thinly to the affected areas.
Results: Most of the pimples disappeared and the pimples that did not were reduced in size.

### Subject F

Subject F
Gender: Female
Age: 51
Skin: Caucasian
Symptoms: Redness of skin, dry skin, sun spots and irritation (adverse reaction to laser therapy treatment)
Treatment: The composition was applied daily to the affected area. The composition was applied liberally to the affected areas.
Results: Following application of the composition redness of the skin subsided, and the sun spots had faded. Subject F noted that her skin was no longer dry.

### Subject G

Subject G
Gender: Female
Age: 26
Skin: Oriental
Symptoms: Cold sores (upper lip)
Treatment: The composition was applied daily to the affected area. The composition was applied liberally to the affected areas.
Results: The cold sore subsided after three days of repeated application and completely disappeared after the fourth day of repeated application. Subject G also noted that the there was no visible scarring.

### Subject G—Trial 1

Subject G
Gender: Female
Age: 26
Skin: Oriental
Symptoms: Pimple scarring on cheek
Treatment: The composition of the first aspect was applied twice a day to the affected area. The composition was applied thinly to the affected areas.
Results: Pimple scarring disappeared after a week of using the claimed composition.

### Subject G—Trial 2

Subject G
Gender: Female
Age: 26
Skin: Oriental
Symptoms: Sensitive skin, eczema, and redness of skin
Treatment: The composition was applied twice a day to the affected area. The composition was applied thinly to the affected areas.
Results: Sensitization of the skin ceased, and the redness of the skin disappeared almost immediately after application of the composition. Subject G had previously used steroid cream to treat her eczema and this resulted in burst capillaries. Subject G notes that the use of the composition did not result in any adverse reactions.

### Subject I

Subject I
Gender: Female
Age: 19
Symptoms: Pimples, redness of the skin
Treatment: The composition was applied twice to the face. The composition was applied liberally to the large pimples and thinly for the smaller pimples.
Results: The pimples decreased roughly half in size after the first application and after the second application the pimples disappeared without scarring.

### Subject J

Subject J
Gender: Female
Age: 33
Skin: Caucasian
Symptoms: Burns, pimples, skin acne and sensitivity.
Treatment: The composition was applied 2-4 times a day to the affected area depending on the severity of the symptoms. The composition was applied thinly to the acne and scars, and liberally to the burns.
Results: The acne, scars and burns were completely healed.

Subject J also applied the composition to her infant child, who suffered from nappy rash with the symptoms of itchiness and swelling. Subject J applied the composition liberally to the affected areas of her child and notes that itching and swelling disappeared within 24 hours.

### Subject K

Subject K
Gender: Female
Age: 2
Skin: Asian
Symptoms: Burns, redness, blistering and swelling to hands and fingers.
Treatment: An application of the composition was applied to her hands every two hours. Her hands were then covered to ensure the composition was not removed. The composition was initially applied liberally to the affected area and the amount applied progressive decreased with the disappearance of the symptoms.
Results: The swelling and redness of the skin disappeared. Relief from pain was achieved approximately 30 minutes after the application of the composition. No scarring was observed as a result of the burns.

### Subject L

Subject L
Gender: Male
Age: 47
Skin: Asian
Symptoms: Burns to face (approximately 30%)
Treatment: The composition was applied every 3-4 hours to the affected area. The composition was applied liberally to the burnt areas.
Results: The burns were completely healed after 2 months of repeated application without any visible scarring.

The combination of macadamia oil, beeswax and shea butter provides a synergistic composition that can reduce, alleviate and/or prevent inflammation and promote wound healing. A further advantage of this combination is that beeswax and shea butter provide humectant properties which can act as a moisturizer. This is particularly advantageous when used topically.

The humectant properties are particularly advantageous because adverse reactions to trauma can include dry skin. The humectant property will alleviate this adverse reaction by ensuring that the skin is moisturized to aid wound healing.

### Further Applications of the Composition

Subject M was a female suffering from severe acne. Subject M had severe acne throughout their teenage to adult life, and had attempt many different products to address this issue. Subject M was treated with the composition described herein. The composition was repeatedly applied nightly. Subject M saw results within one week of repeated application. Subject M remarked on the improvement seen within such a short timeframe.

Subject N was young male who burnt his fingers on the stove. Subject N's hands were treated with the composition described herein. Subject N subsequently was able to forget that they had burned their hands within 3-4 hours of application. Subject N's father remarked how quickly the composition had an effect.

Subject O was an adolescent male who was stung on his back by a jellyfish. The back of Subject O immediately became red and inflamed, and he experienced severe pain. The composition described herein was applied to the affected areas. Subsequently, pain subsided within 15 minutes of application of the composition. After 2 hours post application, the back of Subject O looked normal and Subject O experienced no further pain. Within 4 hours of application, Subject O had forgotten about the jellyfish sting. The stings were healed within a few days.

Subject P was a female who burnt her hands whilst cooking. Application of running water over the affected area resulted in the skin peeling off the hand. The composition described herein was applied to the affect area, and Subject P ensured that the composition was applied overnight. Subject P remarked that, within a week of repeated application of the composition, she was able to dispense of the bandage. The burns were completely healed within 3 weeks of repeated application.

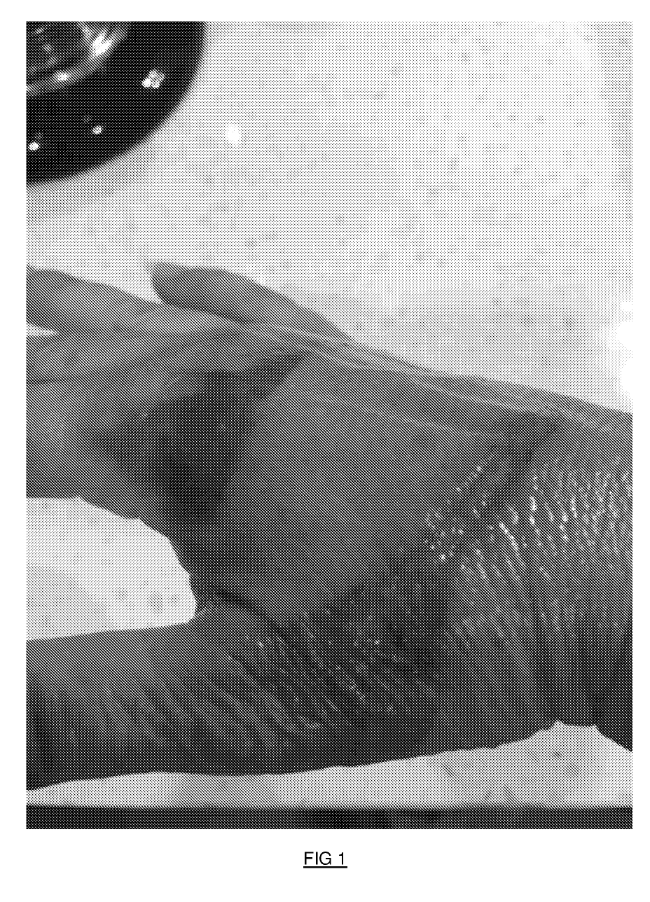

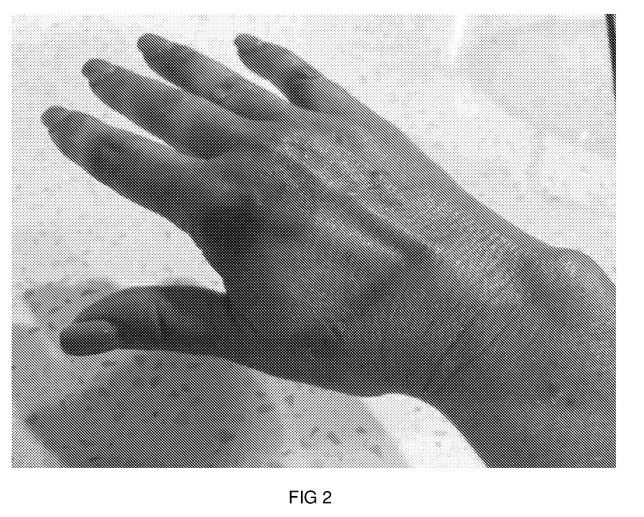

FIG. 1 shows the hand of a patient shortly after a burn, and FIG. 2 shows the same hand after the application of the present composition. It should be clear from these figures that the application of the composition significantly reduced the amount of inflammation from the burn. As stated above, the patient's hand had healed sufficiently to remove the bandage.

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. Accordingly, this invention is intended to embrace all alternatives, modifications and variations of the present invention that have been discussed herein, and other embodiments that fall within the spirit and scope of the above described invention.

1. A composition for treating inflammation and/or promoting wound healing comprising macadamia oil, beeswax and shea butter as the major active components.

2. The composition of claim 1, wherein the % by weight amount of macadamia oil in the composition is between about 65% and about 95%.

3. The composition of claim 1, wherein the % by weight amount of beeswax in the composition is between about 5% and about 15%.

4. The composition of claim 1, wherein the % by weight amount of shea butter in the composition is between about 1% and about 8%.

5. The composition of claim 1 further comprising at least one additional component selected from the group consisting of vitamin E, calendula oil, vitamin B5, benzoin oil, carrot seed oil, patchouli oil, geranium oil, lavender oil, sandalwood oil, rose oil, myrrh oil and citrus oil.

6. The composition of claim 5, wherein the composition further comprises at least 2, or at least 3, or at least 4, or at least 5, or at least 6, or at least 7, or at least 8, or at least 9 of the additional components.

7. The composition of claim 5, wherein the % by weight of the additional components in the composition is less than about 10%, or less than 5%, or less than 2%.

8. The composition of claim 1 comprising, in % by weight amounts of the composition, about 70% to about 90% macadamia oil, about 7% to about 13% beeswax, and about 2% to about 5% shea butter.

9. The composition of claim 5, where the composition comprises macadamia oil, beeswax, shea butter, vitamin E, calendula oil, vitamin B5, benzoin oil, carrot seed oil, patchouli oil, geranium oil, lavender oil, sandalwood oil, rose oil, myrrh oil and citrus oil.

10. The composition of claim 5 comprising, in % by weight amounts of the composition, about 80% macadamia oil, about 10% beeswax, about 4% shea butter, about 1.8% vitamin E, about 0.9% calendula oil, about 0.9% vitamin B5, about 0.7% benzoil oil, about 0.4% carrot seed oil, about 0.4% geraniuim oil, about 0.4% sandalwood oil, about 0.4% rose oil, about 0.4% myrrh oil, and about 0.3% citrus oil.

11. The composition of claim 1, further comprising a preservative.

12. The composition of claim 1, wherein the composition is a topical composition.

13. The composition of claim 1, wherein the composition is a substantially homogenous cream or lotion.

14. A method of treating inflammation and/or promoting wound healing in a subject, the method including the step of administering to the subject a therapeutically effective amount of a composition comprising macadamia oil, beeswax and shea butter as the major active components.

15. The method of claim 14, wherein the step of administering the composition is a step of topical administration.

16. The method of claim 14, wherein the composition is in the form of a topical solution, lotion, shake solution, cream, ointment, gel, foam, transdermal patch, powder, solid, sponge, tape, vapour, paste, or balm.

17. A method of formulating a composition including the steps of mixing macadamia oil, beeswax and shea butter to form the composition wherein the macadamia oil, beeswax and shea butter form the major active components.

18. The method of claim 17, further including the step of adding and mixing at least one additional component selected from the group comprising vitamin E, calendula oil, vitamin B5, benzoin oil, carrot seed oil, patchouli oil, geranium oil, lavender oil, sandalwood oil, rose oil, myrrh oil and citrus oil.

19. A medicament for treating inflammation and/or promoting wound-healing comprising the composition of claim 1.
